(19) United States
(12) Patent Application Publication
(10) Pub. No.: US 2018/0078714 A1
(43) Pub. Date: Mar. 22, 2018

(54) INJECTION NEEDLE

(71) Applicant: NANBU PLASTICS CO., LTD., Shizuoka (JP)

(72) Inventors: Hiroshi OZAWA, Shizuoka (JP); Akinori INOU, Shizuoka (JP); Nobuyuki TAKAHASHI, Shizuoka (JP); Akihiro SHINODA, Shizuoka (JP); Eiji KANI, Shizuoka (JP)

(21) Appl. No.: 15/690,111

(22) Filed: Aug. 29, 2017

(30) Foreign Application Priority Data
Sep. 20, 2016 (JP) 2016-4583

(51) Int. Cl. A61M 5/32; A61M 5/34

(52) U.S. Cl. CPC A61M 5/3298; A61M 5/349

(57) ABSTRACT

An injection needle is provided that allows design of the size of a needle base without being limited by the size of a syringe connecting part. As a hub, a needle base that is formed to have a through hole into which a needle tube is inserted, and a tubular housing where both ends in the axial direction are opened and the needle base is fitted and stored inside of one end, are formed as separate injection molded parts, thereby allowing the needle base to be largely designed. A needle tube shield part is in a box-lid shape, and the back side of a ceiling part thereof is provided with a tubular guiding part. An edge surface thereof abuts on the needle base, and a side part annularly holds and abuts on the housing. Accordingly, the needle base does not move upon receiving the pressure of a drug solution.

### BACKGROUND

### Technical Field

The present disclosure relates to an injection needle for transdermally administering a drug solution.

### Background Art

Injection has been used for a long time to administer drug solutions that cannot be orally administered. Drug solutions are often injected with one needle tube, and since administration by puncturing with an edge of a needle tube involves pain, reduction of pain has been recognized as an objective.

In this regard, from recent years, a type of injection needle that comprises a plurality of needle tubes is proposed in Japanese National Phase PCT Laid-open Publication No. 2013-539990 and the like. By disposing a plurality of thin needle tubes instead of one thick needle tube, reduction of pain due to achievement of a smaller cross-sectional area of needle tubes is expected.

Meanwhile, recently, drug solutions having a high viscosity such as hyaluronic acid have also been used as an object to be injected. Since such a drug solution could not pass through thin needle tubes, a thick needle tube is used as before, and thus measures taken to reduce pain were insufficient.

In addition, such a drug solution is more difficult to be dispersed in intradermal tissue, and it has a tendency to remain in the injection site, i.e., a narrow area centering on the edge of the needle tube. Thus, dispersion of a drug solution in tissue was insufficient by the use of one needle tube.

Furthermore, an injection needle is mainly comprised of a needle tube and a hub that supports the base end side of the needle tube and connects to a syringe. This hub has been conventionally manufactured by an injection molding process of plastic, utilizing a single shaping mold. Accordingly, at the time of designing a needle base, it was impossible to choose to increase the size due to limitation by the unified standard of the size of a syringe connecting part.

Thus, even if spaces between injection sites are broadened in the type of injection needle that supports a plurality of needle tubes to cope with a drug solution that is difficult to be dispersed as stated above, there was a limitation in broadening the spaces between the needle tubes that are arranged in parallel.

In addition, approach to painlessness is an objective that is commonly required for an injection needle. Since a drug solution having a low viscosity can be injected even if the needle tube is made thin, it is expected that pain due to puncturing by a needle tube at the time of injection can be further reduced if the needle tube can be made thinner. In such a case, the total number of needle tubes would be increased. However, as stated above, there is a limitation in designing of the size of a needle base. Thus, there is also a limitation in increasing the number of needle tubes, as long as they are supported by the needle base.

### BRIEF SUMMARY

Embodiments of the present invention are made by focusing on the above-described conventional problem, and the purpose thereof is to provide a novel and useful injection needle that allows design of the size of a needle base without being limited by the size of a syringe connecting part.

In order to achieve the above-described objective, an embodiment of the present invention is directed to an injection needle comprising: a plurality of needle tubes having a discharge opening for a drug solution in the needle tip; a hub that supports each of the plurality of needle tubes at the base end side and connects to a syringe; and a needle tube shield part that is attached to the base end side of the needle tube to shorten the length of the projected tip of the needle tube, wherein the hub is configured in a state divided into a needle base that is formed to have through holes into which the needle tubes are inserted, and a tubular housing where both ends in the axial direction are opened and the needle base is fitted and stored in the inside of one end in the axial direction, wherein the housing and the needle base are connected by an adhesive that infiltrates from the needle tip side into a fitting gap formed therebetween, or by welding, wherein the needle tube shield part is in a box-lid shape, and the back side of a ceiling part thereof is provided with guiding parts that surround the needle tubes for guiding, and wherein the guiding parts abut on the needle tubes projecting surfaces of the needle base, and the side part annularly holds and abuts on the housing.

Another embodiment of the present invention is directed to an injection needle as described above, wherein, when seen from the puncture/removal direction of the needle tube, the outline of the cross section of the needle base is positioned to the outer side than the inner outline of the cross section of the syringe connecting side of the housing.

Another embodiment of the present invention is directed to an injection needle as described above, wherein an opening on the needle tip side of a fitting gap is expanded by the retraction of the needle base side to form an annular concave part to act as an entrance to the fitting gap and a filling portion for an adhesive.

Another embodiment of the present invention is directed to an injection needle as described above, wherein the side part of the needle tube shield part extends to the entry site of an adhesive or a melt fluid, which is generated at the time of welding, into the fitting gap for holding and abutting.

Another embodiment of the present invention is directed to an injection needle as described above, wherein the guiding part is configured to be a tubular body.

Another embodiment of the present invention is directed to an injection needle as described above, wherein the needle base and the housing are both injection molded parts.

### Effect of the Invention

According to embodiments of the injection needle of the present invention, the size of a needle base can be designed without limitation of the size of a syringe connecting part, and by achieving a larger size of the needle base, it is possible to broaden the spaces between a plurality of needle tubes that are arranged in parallel, or increase the number of needle tubes.

In addition, embodiments of the injection needle of the present invention can be manufactured by injection molding as in the case of a conventional one by utilizing two shaping molds.

### BRIEF DESCRIPTION OF THE SEVERAL VIEWS OF THE DRAWINGS

FIG. 1 is an entire perspective view of a five-needle type injection needle defined by an embodiment of the present invention.

FIG. 2 is an exploded perspective view of the injection needle of FIG. 1.

FIG. 3 is a halved perspective view of the injection needle of FIG. 1.

FIG. 4 is a side view of the injection needle of FIG. 1.

FIG. 5 is a cross-sectional view in the direction corresponding to FIG. 4.

FIG. 6 is a cross-sectional view in the vertically intersecting direction of FIG. 5.

FIG. 7 is an exemplary view of action of force that works at the time of injection, based on FIGS. 5 and 6.

FIG. 8 is a view showing an exemplary modification of the needle base of the injection needle of FIG. 1.

FIG. 9 is an entire perspective view and a halved perspective view of a nine-needle type injection needle that is different from that of FIG. 1.

### DETAILED DESCRIPTION

An injection needle 1 defined by an embodiment of the present invention will be explained in accordance with the drawings.

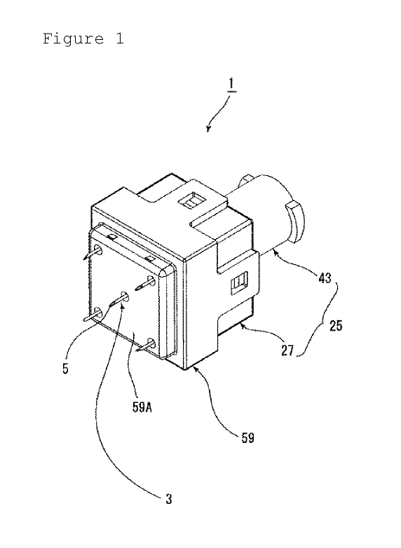

As shown in FIG. 1, the injection needle 1 is a five-needle type, and it is provided with five needle tubes 3 having the same diameter and that are linear.

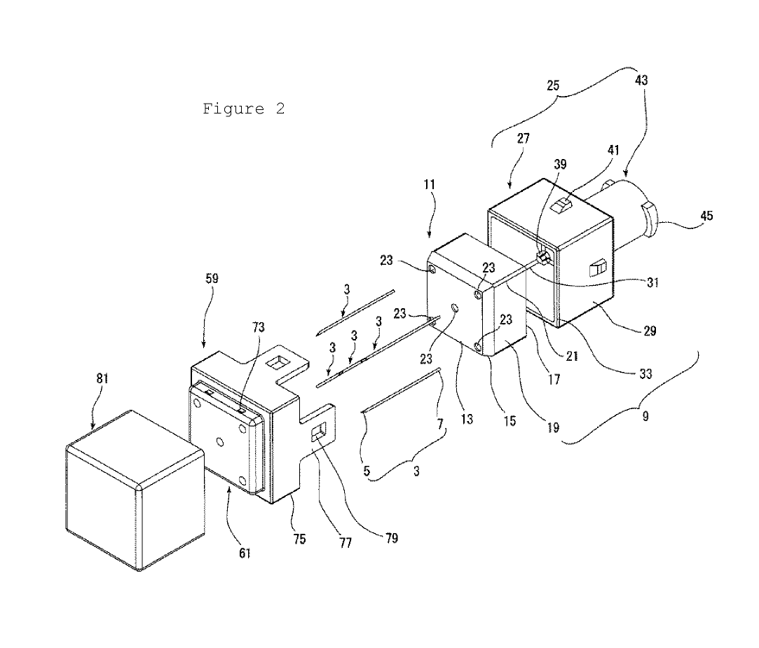

In each needle tube 3, as shown in FIG. 2, a needle tip 5 is formed as a discharge opening for a drug solution, and a base end 7 is supported by a hub 9 to be integrated. This hub 9 is comprised of two injection molded parts.

One of the injection molded parts is a needle base 11. This needle base 11 is shaped in a block state that is substantially a square shape, and the square-shaped surface on the side to which the needle tip 5 of the needle tube 3 protrudes, is a front surface 13. The whole circumference of the edge part of this front surface 13 is uniformly chamfered to from a inclined surface 15, and the front surface 13 is in a similar scale down shape of a back surface 17 by being scaled down just as much as the chamfered portion. The front surface 13 and the back surface 17 are in parallel, and four side surfaces 19 are exposed in the vertically intersecting direction therebetween. The corner parts at the boundaries of the adjacent side surfaces 19 are all provided with a roundness 21.

From the front surface 13 to the back surface 17, five through holes 23 which penetrate the needle base 11 are formed. Each through hole 23 linearly extends in the direction that vertically intersects with the front surface 13 and the back surface 17. The cross-section surface in the vertical direction thereof is in a circular shape, and it has the same diameter except for the opening end which opens to the front surface 13. Furthermore, the opening end side is expanded in a tapered state, and it is an entrance and a reservoir for an adhesive.

Accordingly, five through holes 23 are arranged in a state that they are parallel with one another, and among the five through holes 23, one through hole is opened at the center position of the front surface 13, and other four are opened at locations closer to four corner parts of the front surface 13. Five through holes 23 each match the above-described five needle tubes 3, and one needle tube 3 is inserted in each through hole 23. The center of the through hole 23 opened in the center position is the center in the axial direction of the entire injection needle 1, and this axial direction is the puncture/removal direction of the needle tube 3.

Hereinafter, explanations will be made by utilizing this “axial direction” as the standard index of directions. In addition, one end of the axial direction will be expressed as the “puncture side of the axial direction,” and the other end will be expressed as the “removal side of the axial direction.”

The other of the injection molded parts is a housing 25. This housing 25 is formed in a tubular shape, and the axial direction thereof matches with the center of the axial direction of the injection needle 1. Both ends in the axial direction are opened.

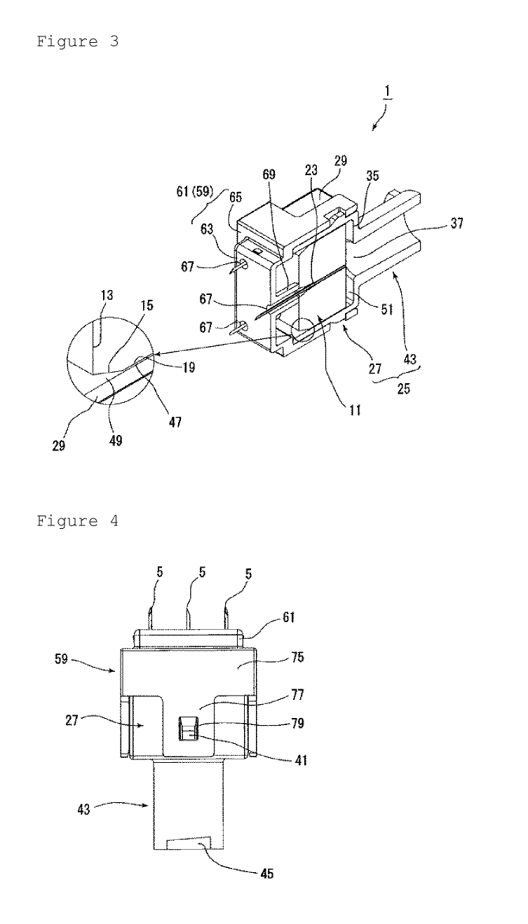

As shown in FIGS. 3 and 4, the puncture side of the axial direction is a fitting storage part 27 of the needle base 11.

This fitting storage part 27 is configured in a hollow square shape by the connection of the thickness sides of four rectangular plate side parts 29 and the vertical section in the axial direction, where the thickness sides can be seen, forms a substantially square frame. The corner part in the inner surface is provided with a rounded depression 31. This frame size is the same size in any vertical section in the axial direction.

The edge on the puncture side of the axial direction of the fitting storage part 27 is chamfered. However, a flat edge surface 33 having a certain amount of area is left in the inward side. This edge surface 33 is on the vertical section in the axial direction.

As shown in FIG. 3, from the edge on the removal side, a plate-like inward flange 35 inwardly extends and narrows the opening. A communication is established in the axial direction via a communication hole 37 in a circular shape, which is left at the center. In each of the corner parts at four corners blocked with the inward flange 35, a stopping convex part 39 that is in a square shape is formed.

In addition, as shown in FIG. 2, a hook projection 41 is formed on each of the external surfaces of the four side parts 29.

On the other hand, the removal side of the axial direction is a syringe connecting part 43.

The vertical section in the axial direction of this syringe connecting part 43 is in a circular shape, and it is formed to have the same diameter along the axial direction. The inner diameter of the syringe connecting part 43 is substantially the same as the communication hole 37, and this is continuous with the fitting storage part 27 by having the inward flange 35 as the stepped part.

The edge on the removal side is fully opened, and the outside surface thereof is formed to have a connecting convex part 45 for syringe connection.

The needle base 11 is fitted and stored in the internal space of the fitting storage part 27 in a nested-state, in a posture where the front surface 13 is exposed towards the puncture side of the axial direction. Thus, the roundness 21 in the corner part faces the depression 31 of the fitting storage part 27.

The needle base 11 is loosely fit in the fitting storage part 27, and as shown in FIG. 3, although a fitting gap 47 is formed between the side surface 19 of the needle base 11 and the inner surface of the side part 29 of the fitting storage part 27, and also between the roundness 21 and the depression 31, this is a slight gap. Thus, a relative rotation around the axis is not possible.

Furthermore, an expanded annular concave part 49 is formed between the inclined surface 15 and the inner surface of the side part 29 on the fitting storage part 27 side. The annular concave part 49 is formed such that the vertical section in the axial direction is in a triangular shape, and it is continuous with the fitting gap 47.

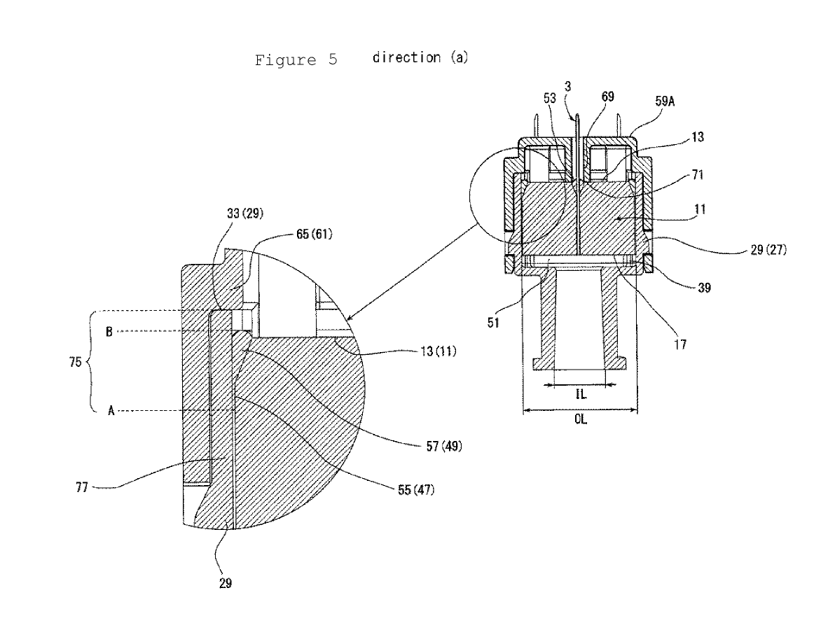

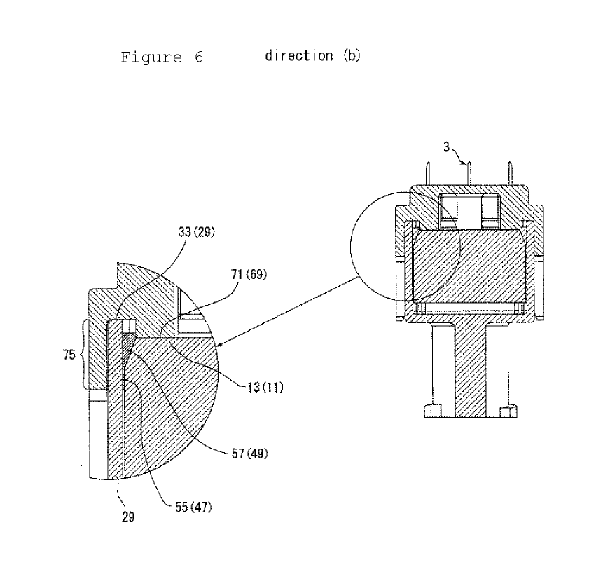

As shown in FIGS. 5 and 6, the length of the fitting storage part 27 in the axial direction is designed to be longer than the length of the needle base 11 in the axial direction, and the back surface 17 of the needle base 11 is embedded to a depth that abuts on the stopping convex part 39 (FIG. 2) to be stopped there. Accordingly, a space 51 for guiding a drug solution is left between the back surface 17 and the inward flange 35. This space 51 is broadened in the radially outward direction more than the flange connecting part 43, and the broadened site is a portion that becomes undercut when the needle base 11 and the fitting storage part 27 are regarded as a single injection molded part.

On the other hand, the front surface 13 of the needle base 11 is in a state that is one stage lower than the edge surface 33 of the fitting storage part 27.

Reference numerals 55, 57 show joint parts that are fixed. As the joining technique, not only adhesion by an adhesive, but so-called welding is also applicable. The joint parts 55, 57 are comprised of such adhesive.

The curing type of the adhesive can be a room temperature curing adhesive, a thermosetting adhesive, or UV-curable adhesive.

However, the joint part is preferably a thermosetting adhesive or UV-curable adhesive.

These adhesives have been conventionally used for adhesion and fixation of a needle tube to a hub. The adhering part 53 is interposed for adhesion of the needle tube 3, which is inserted in the through hole 23 of the needle base 11, against the needle base 11. The adhesive is filled by having the opening end side of the through hole 23 as its entrance, and the adhesive deeply penetrates into the gap between the needle tube 3 and the through hole 23 by capillary action. The opening end side eventually becomes a reservoir where the adhesive is swelled in a bump state and accumulated.

The needle tube 3 that is used, is sufficiently long. Thus, when filling an adhesive, it is easy to support the needle tube 3 in an inserted state, and the adhesive will not touch the needle tip 5.

The adhering part 55 is interposed for adhesion and fixation of the needle base 11 against the fitting storage part 27. The annular concave part 49 is an entrance of an adhesive, wherein the adhesive infiltrates into the fitting gap 47 and is solidified. The depth of penetration of an adhesive into the fitting gap 47 exceeds half the length of the needle base 11, and the end thereof is point “A.” In addition, an adhesive is swelled in a bump state at the annular concave part 49, and the end thereof is point “B.” The adhering part extends from the fitting gap 47 to the annular concave part 49, and it is interposed between the needle base 11, which is the inward side, and the fitting storage part 27, which is the outward side, in an inner tube state to adhere the two with a large adhesive area.

For convenience of explanation, the joint part may be distinguished between the joint part 55 on the fitting gap 47 side and the joint part 57 on the annular concave part 49 side. However, a seamless and continuous joint part is formed, and the flow line is in succession.

As stated above, the needle tubes 3 are fixed in a manner that the base end 7 is supported against the needle base 11, and the needle base 11 is fixed against the housing 25 in the above-described manner to be integrated. Furthermore, as shown in FIG. 1, a needle tube shield part 59 is attached to the needle base 11 side.

This needle tube shield part 59 is also an injection molded part, and it has a box-lid shape.

As shown in FIGS. 2 and 3, a ceiling part 61 is in the form of a plate having a square shape, and has a two-stage structure where a four-side peripheral part 65 is made one stage lower with respect to a central part 63. This central part 63 is formed to have five through holes 67 where the cross section is a circular shape, and a tubular guiding part 69 is continued at the back surface along the hole edge of each through hole 67. This tubular guiding part 69 has the same diameter and is linear.

Among the five through holes 67, one through hole is formed at the center position of the central part 63, and other four are each formed closer to the four corner parts of the central part 63, as in the case of the through hole 23 on the front surface 13 side of the needle base 11. As shown in FIGS. 5 and 6, each tubular guiding part 69 extends beyond the ceiling part 61 which has a step, and the edge surface 71 is a flat surface that is in parallel with the plate surface of the ceiling part 61.

In addition, as shown in FIG. 2, on the outer surface of the ceiling part 61, pressing convex parts 73 in pairs are provided in two opposing surfaces, and each pressing convex part 73 is formed in a state that is outwardly projected.

With respect to the ceiling part 61, four plate-like side parts 75 are vertically raised, and tongue pieces 77 are created to extend from each of them. Each tongue piece 77 extends from the central portion of the end edge of the side part 75, and a hooking hole 79 in a square shape is formed therein.

As shown in FIG. 4, the needle tube shield part 59 is covered on the square-shaped fitting storage part 27 as a square-shaped box lid to be integrated, and the side parts 75 surround the side parts 29 of the fitting storage part 27 from the outside, thereby allowing the hook projection 41 on the fitting storage part 27 side to be hooked and locked in the hooking hole 79 of the needle tube shield part 59 side. Accordingly, relative movements of the needle tube shield part 59 in the axial direction and the direction around the axis with respect to the fitting storage part 27, and hence housing 25, are restricted.

In this covered state, one tubular guiding part 69 corresponds to the axial direction of one needle tube 3, and the base end 7 side of the needle tubes 3 is shielded by the needle tube shield part 59, thereby allowing the length of the projected tip to be short. As stated above, the needle tube 3 projects from the front surface 13 of the needle base 11 by a certain length such that the operation is facilitated when fixing to the needle base 11 with an adhesive. In this regard, this needle tube shield part 59 is utilized and the front surface of the needle tube shield part 59 is set as a reference plane 59A of puncture depth as shown in FIGS. 5 and 6, to allow accurate injection to a predetermined depth in tissue.

In addition, in this covered state, the peripheral part 65 of the ceiling part 61 abuts on the edge surfaces 31 of the side parts 29 of the fitting storage part 27, and the side part 75 abuts and holds from the outside. The side part 75 has a certain length, and it extends to around point A of the adhering part 55 entered into the fitting gap 47.

In addition, the edge surface 71 of each tubular guiding part 69 abuts on the front surface 13 of the needle base 11. One edge surface 71 is positioned at the center of the front surface 13, and four edge surfaces 71 are uniformly disposed in the surrounding of the front surface 13. Since the edge surface 71 closer to the corner part does not span the inclined surface 15 of the needle base 11, it does not contact the adhering part 57 which is in a bump state, and the entirety thereof abuts on the front surface 13.

An embodiment of the injection needle 1 of the present invention is configured in the above-described manner, and when seen from the puncture/removal direction of the needle tube 3, the outline (OL) of the cross section of the needle base 11 is positioned to the outer side than the inner outline (IL) of the cross section of the syringe connecting side of the housing 25.

In other words, the size of the needle base 11 is increased without being limited by the size of the syringe connecting part, and five needle tubes 3 are disposed with a sufficient space between each other, on the front surface 13 of the needle base 11.

Furthermore, as shown in FIG. 2, at the time of storage, a cap 81 is covered on the needle tip 5 side of the needle tube 3. The pressing convex parts 73 made in the ceiling part 61 are pressed by the inner surface of the cap 81 to be closed.

The length of the projected tip of the needle tube 3, i.e., the length until the needle tip 5 of the needle tube 3 by setting the exposed surface of the ceiling part 61 of the needle tube shield part 59 as a reference, is from 0.05 to 3.5 mm.

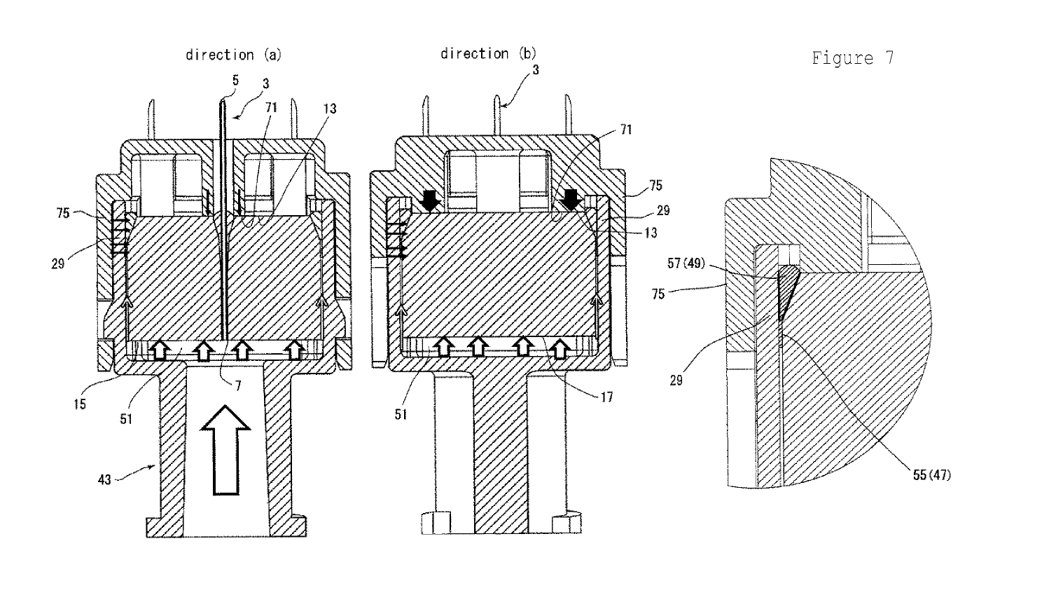

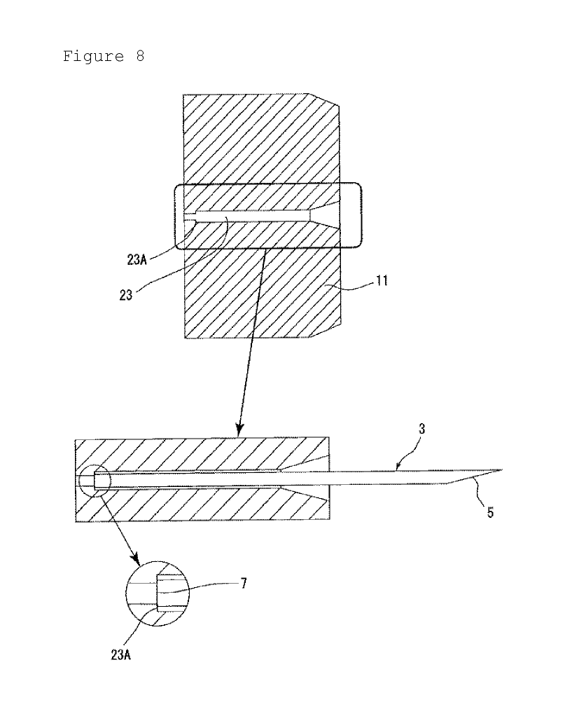

The influence of pumping of a drug solution at the time of injection is shown in FIG. 7.

At the time of injection, when a drug solution is infused to the syringe connected to the injection needle 1 and is pressed by the plunger, the drug solution is pumped from the syringe connecting part 43 of the housing 25 towards the fitting storage part 27.

Furthermore, when the drug solution reaches each of the base ends 7 of five needle tubes 3, it enters into each of them to be discharged from the needle tips 5.

The drug solution firstly enters into the space 51 from the syringe connecting part 43, and it is distributed and fed to each needle tube 3 therefrom upon receiving pressure. Thus, as shown in white arrows, the drug solution reached around the back surface 17 side of the needle base 11 tries to push up the needle base 11 from the fitting storage part 27.

In contrast, since the edge surface 71 of the tubular guiding part 69 of the needle tube shield part 59 abuts on the front surface 13 of the needle base 11, it tries to push back as shown in black arrows. Since the edge surfaces 71, are uniformly disposed in the center of the axis and the surrounding thereof, the power is balanced in the axial direction, and no moment is generated.

In addition, the adhering part 57 which is buried in the annular concave part 49 is in a bump state and adheres to the inclined surface 15 of the needle base 11 so as to increase the adhesive area. At the same time, as shown in the black arrows, the side part 75 of the ceiling part 61 holds the side part 29 of the fitting storage part 27 to apply force that suppresses swelling to the outside. Thus, the adhesive strength of the thick-line portion shown in the enlarged view is effectively reinforced. Accordingly, this adhering part 57 also cooperates to effectively resist the above-described pressure to push up.

In addition, the drug solution that reached around the fitting gap 47 tries to cleave the adhering part 55 filled in the fitting gap 47 between the side part 29 of the fitting storage part 27 and the needle base 11. However, also regarding the fitting gap 47, as shown in the black arrows, the side part 75 of the ceiling part 61 holds the side part 29 of the fitting storage part 27 to apply force that suppresses swelling to the outside. Further, the above-described adhering part 57 exerts a strong damming effect. Accordingly, the above-described pressure to cleavage is effectively resisted.

In embodiments of the present invention, when a drug solution is pumped, the needle base 11 receives pressure in the direction to be pushed out from the fitting storage part 27. However, as stated above, by devising the structures of the needle base 11, fitting storage part 27, and needle tube shield part 59, and the assembled state thereof, the relative position of the fitting storage part 27 to the needle base 11 is accurately maintained, and a pressure drop during injection is prevented.

In addition, the guiding part of the needle tube 3 is formed to have a tubular structure in the needle tube shield part 59, thereby achieving weight reduction.

The drug solution used in embodiments of the injection needle 1 of the present invention is typically a solution, gel or suspension that contains the drug solution. Drugs that can be used are substantially not limited as long as they are not such drugs unsuitable for transdermal administration.

Major drugs can be, for example, hyaluronic acid, collagen, botox or the like, antibacterial drug, antiviral drug, vaccine, antineoplastic drug, immunosuppressant, steroid drug, anti-inflammatory drug, antirheumatic drug, arthritis therapeutic agent, antihistaminic drug, antiallergic drug, antidiabetic drug, hormone drug, bone/calcium metabolic drug, vitamin, blood preparation, hematopoietic drug, antithrombotic drug, antihyperlipidemic drug, antiarrhythmic drug, vasodilator, prostaglandin, calcium antagonist, ACE inhibitor, β-blocker, antihypertensive drug, diuretic drug, xanthine derivative, β agonist, antiasthmatic drug, antitussive drug, expectorant drug, anticholinergic drug, antidiarrheal drug, stomachic digestant, antiulcer drug, purgative drug, sleeping pill, sedative, antipyretic, cold medicine, antiepileptic drug, antipsychotic drug, antidepressant drug, antianxiety drug, central nervous system stimulant, parasympathomimetic drug, sympathomimetic drug, antiemetic drug, central stimulant, antiparkinsonian drug, muscle relaxant drug, anticonvulsant, anesthetic, antipruritic drug, antimigraine drug, diagnostic agent, oligonucleotide, genetic drug, and the like.

However, the drugs are preferably proteins, peptides, polysaccharides, oligonucleotides, DNAs and the like that do not exert effects or that are weakened in oral administration. Specifically, the drugs are high-molecular weight pharmaceutical products such as insulin, growth hormone, interferon, calcitonin and the like.

The embodiments of the present invention have been explained above. However, the specific configurations of the present invention are not limited to the above-described embodiments, and design changes that do not deviate from the gist of the present invention are also included in the present invention.

For example, as shown in FIG. 8, a step 23A can be provided in the through hole 23 of the needle base 11 so that the base end 7 of the needle tube 3 is stopped at that step 23A.

In addition, since the reference plane 59A of the needle tube shield part 59 is made large, it can be considered that this could be curved along the shape of the surface of the site to be injected.

Furthermore, although the front surface 13 of the needle base 11 has a square shape, it can be made into a circular shape and the like.

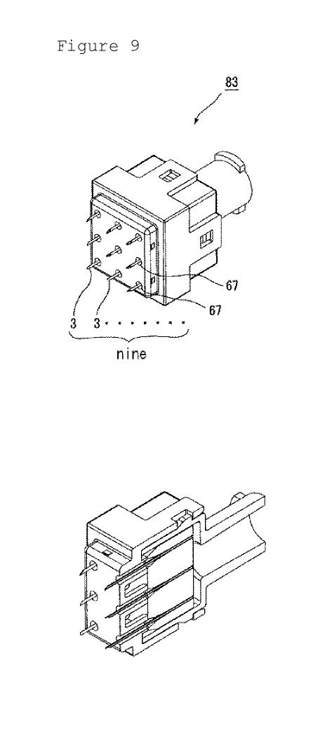

FIG. 9 shows an injection needle 83, which is another example. This injection needle 83 is a nine-needle type. In this manner, the size of the needle base can be designed without being limited by the size of the syringe connecting part. Thus, it is possible to increase the number of needle tube 3 than the conventional ones.

In addition, it is also possible to widen the pitch of the needle tube 3, and the number of the needle tube 3 can be increased from 2 to 36 needle tubes.

In addition, not only adhesives, but a welding technique can also be utilized for the joining parts 55, 57. In that case, the fitting gap 47 is filled with a fluid entered from the needle base 11 and the fitting storage part 27 surrounding the fitting gap 47 by melt flow, and a matter corresponding to the adhering part 55 is formed. Further, although the annular concave part 49 is generally not formed on the opening end side, a matter corresponding to the adhering part 57 is formed after welding. Accordingly, the same effect as in the case of using an adhesive can be enjoyed.

The types of welding can be laser welding, ultrasonic welding, vibration welding, and heat welding.

This application claims priority to Japanese Utility Model Application No. 2016-4583, filed Sep. 20, 2016, the entire contents of which are hereby incorporated by reference.

Aspects and features of the various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

1. An injection needle comprising a plurality of needle tubes and a hub for supporting the plurality of needle tubes, wherein:
the hub comprises a needle base having a plurality of through holes into which the plurality of needle tubes are inserted and a housing for storing the needle base, wherein the needle base and the housing are joined;
the housing comprises a syringe connecting part on the base end side of the housing, and the syringe connecting part is configured to be connected to a syringe; and
a cross-sectional area of the needle base is larger than a cross-sectional area of the syringe connecting part.

2. The injection needle according to claim 1, wherein:
the injection needle further comprises a needle tube shield part;
the needle tube shield part comprises a ceiling part and a side part;
the plurality of needle tubes are disposed to be projected from the ceiling part of the needle tube shield part; and
the needle tube shield part and the housing are configured such that an inner surface of the side part of the needle tube shield part abuts on an outer surface of the housing.

3. The injection needle according to claim 1, wherein a gap is formed between the needle base and the housing, and the gap is filled with an adhesive or melt fluid.

4. The injection needle according to claim 2, wherein a gap is formed between the needle base and the housing, and the gap is filled with an adhesive or melt fluid.

5. The injection needle according to claim 4, wherein a range where the inner surface of the side part of the needle tube shield part abuts on the outer surface of the housing includes a range where the gap is filled with the adhesive or melt fluid.

6. The injection needle according to claim 2, wherein the needle tube shield part further comprises a plurality of guiding parts for guiding the plurality of needle tubes, and the plurality of guiding parts abut on the front surface of the needle base.

7. The injection needle according to claim 3, wherein: an annular concave part is formed in an outer edge of a tip part of the needle base; an additional gap is formed between the needle base and the housing by the concave part; and the additional gap is further filled with an adhesive or melt fluid.
